(19) United States
(12) Patent Application Publication
(10) Pub. No.: US 2026/0315231 A1
(43) Pub. Date: Oct. 15, 2026

(54) MAMMALIAN BONY IMPLANT AND ANCHORS INSERTER SYSTEM

(71) Applicant: Osseus Fusion Systems, Dallas, TX (US)

(72) Inventors: Chase D. Tipping, Dallas, TX (US); Kyle Blaskovich, Dallas, TX (US)

(21) Appl. No.: 19/645,174

(22) Filed: Apr. 11, 2026

Related U.S. Application Data
(60) Provisional application No. 63/787,258, filed on Apr. 11, 2025.

(51) Int. Cl. A61F 2/30; A61F 2/44; A61F 2/46

(52) U.S. Cl. CPC A61F 2/30749; A61F 2/4455; A61F 2/4611; A61F 2002/30131; A61F 2002/30622; A61F 2002/30774

(57) ABSTRACT

Embodiments of the present invention provides systems and methods for deploying implants and anchors to treat one or more bony segments. A system may enable a User to insert an implant between adjacent bony segments and advance one or more bony anchors thereafter via the same system. Other embodiments may be described and claimed.

### TECHNICAL FIELD

Various embodiments described herein relate generally to treating mammalian bony segments, including systems and methods that help deploy implants and anchors to stabilize, maintain spacing between, or couple one or more mammalian bony segments.

### BACKGROUND INFORMATION

It may be desirable to treat one or more bony segments via mammalian implants and bony anchor(s) to encourage bony fusion, stabilize, maintain spacing between, or couple the bony segments. The present invention provides systems and methods for deploying implants and anchors to treat one or more bony segments.

### BRIEF DESCRIPTION OF THE DRAWINGS

FIG. 1A is a simplified isometric front drawing of a mammalian bony anchor (MBA) according to various embodiments.

FIG. 1B is a simplified isometric rear drawing of an MBA according to various embodiments.

FIG. 1C is a simplified top view of an MBA according to various embodiments.

FIG. 1D is a simplified bottom view of an MBA according to various embodiments.

FIG. 1E is a simplified left side view of an MBA according to various embodiments.

FIG. 1F is a simplified bottom front view of an MBA according to various embodiments.

FIG. 1G is a simplified rear view of an MBA according to various embodiments.

FIG. 1H is a cross-sectional view of left side of an MBA taken at line BB shown in FIG. 1G according to various embodiments.

FIG. 2 is a simplified isometric view of an adjacent mammalian bony segments fixation system including an implant and a plurality of mammalian bony anchors according to various embodiments.

FIG. 3A is a simplified isometric front drawing of an adjacent mammalian bony segments implant that may be employed as an Anterior Cervical Implant (ACI) according to various embodiments.

FIG. 3B is a simplified front drawing of an adjacent mammalian bony segments ACI according to various embodiments.

FIG. 3C is a simplified, enlarged isometric front drawing of an adjacent mammalian bony segments ACI according to various embodiments.

FIG. 3D is a simplified top drawing of an adjacent mammalian bony segments ACI according to various embodiments.

FIG. 4A is a simplified isometric front drawing of another adjacent mammalian bony segments implant may be employed as an Oblique Lateral Interbody Fusion (OLIF) implant according to various embodiments.

FIG. 4B is a simplified isometric side drawing of the other adjacent mammalian bony segments OLIF implant according to various embodiments.

FIG. 4C is a simplified top drawing of the other adjacent mammalian bony segments OLIF implant according to various embodiments.

FIG. 5A is a simplified isometric front drawing of a third adjacent mammalian bony segments implant may be employed as an Oblique Lateral Interbody Fusion (OLIF) implant according to various embodiments.

FIG. 5B is a simplified isometric side drawing of the third adjacent mammalian bony segments OLIF implant according to various embodiments.

FIG. 5C is a simplified top drawing of the third adjacent mammalian bony segments OLIF implant according to various embodiments.

FIG. 6 is a simplified isometric drawing of a fourth adjacent mammalian bony segments implant that may be employed as a Lateral Lumbar Interbody Fusion (LLIF) implant according to various embodiments.

FIG. 7A is an isometric simplified drawing of a combination implant and anchor inserter system (CIAIS) coupled to an implant and anchors according to various embodiments.

FIG. 7B is an isometric simplified enlarged drawing of Area AA shown in FIG. 7A according to various embodiments.

FIG. 7C is an isometric simplified top drawing of the combination implant and anchor inserter system (CIAIS) according to various embodiments.

FIG. 7D is an isometric simplified bottom drawing of the combination implant and anchor inserter system (CIAIS) according to various embodiments.

FIG. 7E is an isometric simplified enlarged drawing of Area CC shown in FIG. 7C according to various embodiments.

FIG. 7F is an isometric simplified drawing of the removable elements of the CIAIS as shown in FIG. 7A according to various embodiments.

FIG. 8A is a simplified side view of straight-line removal tool for a curved MBA according to various embodiments.

FIG. 8B is a simplified side view of variable-angle removal tool for a curved MBA according to various embodiments.

FIG. 8C is a simplified enlarged view of area BB of the variable-angle removal tool for a curved MBA shown in FIG. 8B.

FIG. 8D is a simplified isometric view of slap tool for a curved MBA according to various embodiments.

FIG. 9 is a diagram of algorithm for inserting an AMBSFS via CIAIA according to various embodiments.

### DETAILED DESCRIPTION

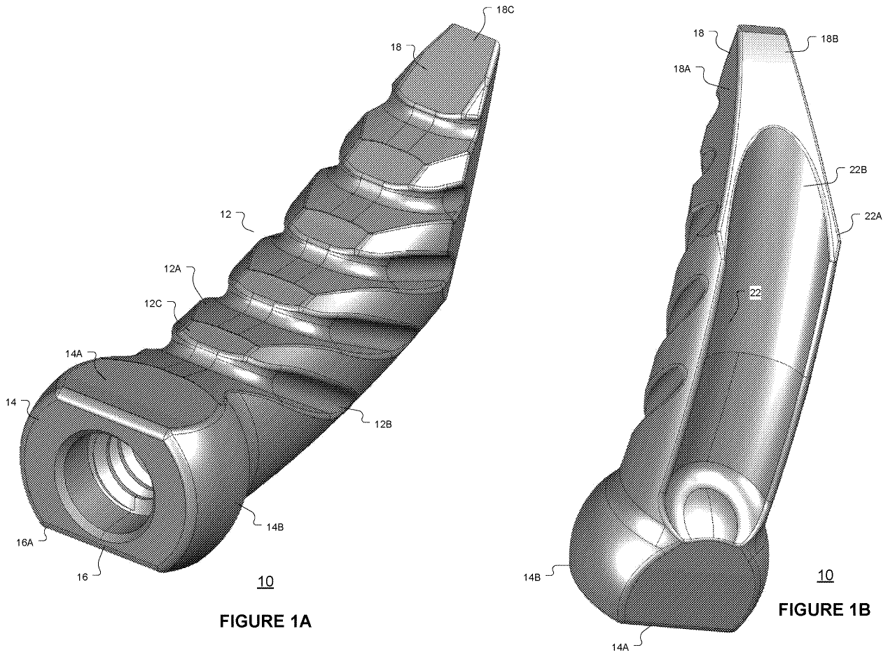

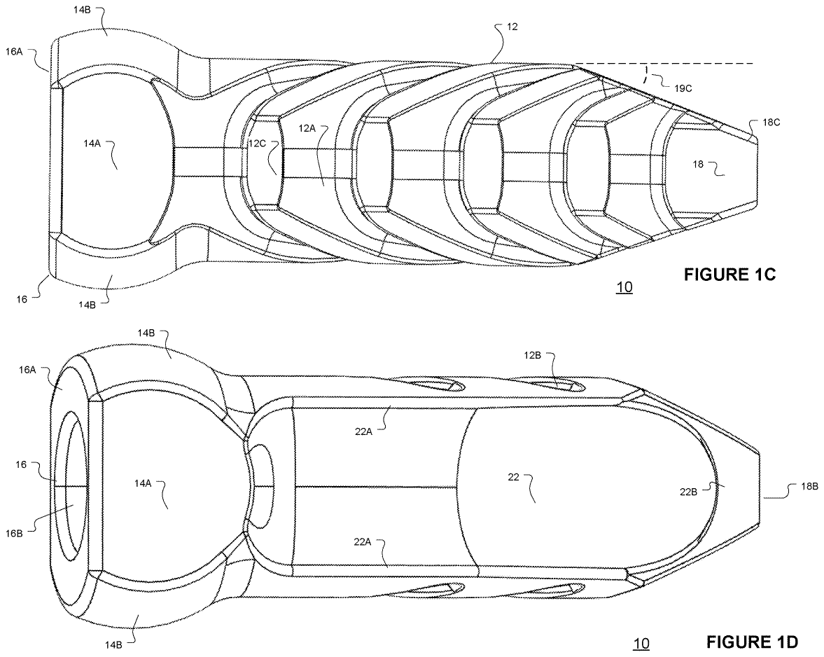

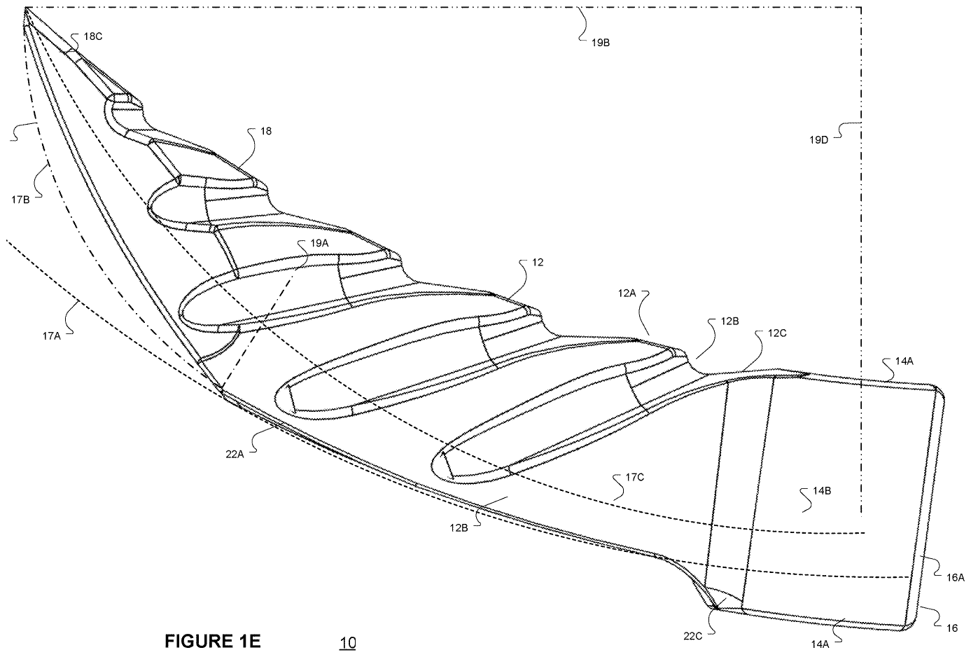

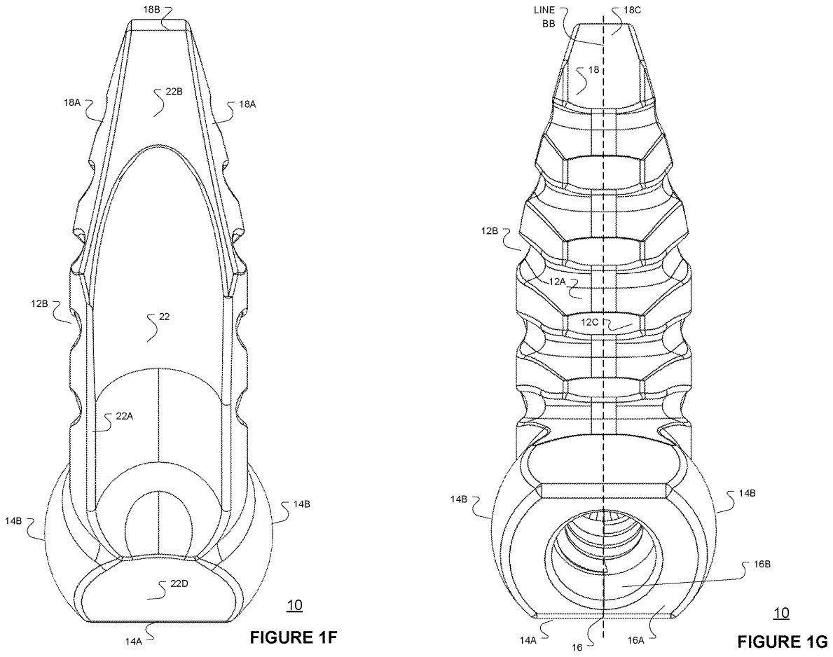

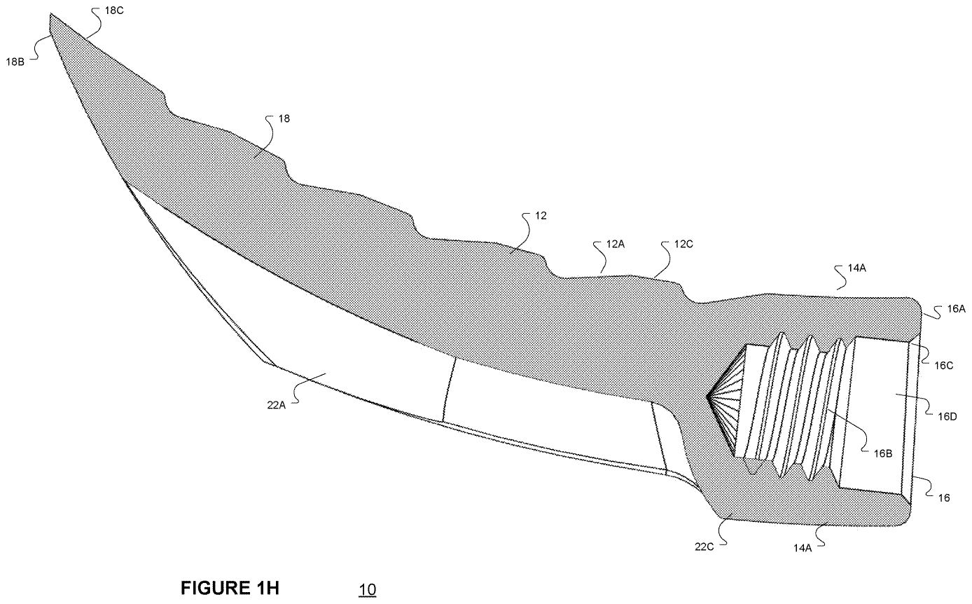

It may be desirable to treat one or more bony segments via mammalian bony anchor(s) 10 such as in conjunction with other system(s) 30A-D to encourage bony fusion, stabilize, maintain spacing between, or couple the bony segments. FIG. 1A is a simplified isometric front drawing of a mammalian bony anchor 10 according to various embodiments. FIG. 1B is a simplified isometric rear drawing of the mammalian bony anchor 10 according to various embodiments. FIG. 1C is a simplified top view of the mammalian bony anchor 10 according to various embodiments. FIG. 1D is a simplified bottom view of the mammalian bony anchor 10 according to various embodiments. FIG. 1E is a simplified left side view of the mammalian bony anchor 10 according to various embodiments. FIG. 1F is a simplified bottom front view of the mammalian bony anchor according to various embodiments. FIG. 1G is a simplified rear view of the mammalian bony anchor 10 according to various embodiments. FIG. 1H is a cross-sectional view of left side of a mammalian bony anchor taken at line BB shown in FIG. 1G according to various embodiments.

As shown in FIGS. 1A-1H, the mammalian bony anchor (MBA) 10 includes a shaft 12, a base section 14, a tool interface 16, and a tapered extended tip section 18. As shown in FIGS. 1E and 1H, the MBA 10 may have an arcuate shape with a primary radius 17C for the anchor 10, a secondary radius 17A for the base section 12, and a third radius 17B for the tip section 18 where the radius 17A is larger than the radius 17C and radius 17B. In an embodiment, the radius 17A may be about 1.5 to 2.5 times larger than radius 17B and about 1.75 to 2.0 times larger in an embodiment, 2.0 to 3.0 times larger than radius 17C. As shown in FIG. 1E, the MBA 10 may have an effective depth of insertion 19B which is less than the overall length 17C of the MBA 10. The MBA 10 may also have a height 19D where the length of the effective hypotenuse formed by 19B and 19D (square root of the sum of the squares of the 19B and 19D) that is less than the overall length 17C of the MBA 10.

As shown in FIGS. 1A-1G, the shaft section 12 and tip section 18 may include a plurality of shelves or scallops 12A. Each scallop 12A may include a flat ledge 12C and an undercut 12B. The flat ledge 12C may ease insertion of the MBA 10 into a bony segment 52A-C while the remainder of each scallop 12A including the edge around the undercut 12B may help prevent expulsion of the MBA 10 once inserted into a desired position in a bony segment.

As also shown in FIGS. 1A-1G, the tip section 18 may also include a large flat region 18C on the top, flat portions 18A on the sides, and a smaller flat region 18B on the bottom that may also ease insertion of the MBA 10 into a bony segment. As also shown in FIGS. 1A-1G, the tip section 18 may have a narrower tip where the sides 18A may form an angle (19C in FIG. 1C) of about 20 to 60 degrees and about 40 degrees in an embodiment. As also shown in FIGS. 1A-1G, the bottom 22 of the MBA 10 may be partially cylindrical in relief, with smaller sections 22B in the tip section 18 than the sections 22A of the rear in the shaft section 12. The shape of the rear 22 may also reduce the force required to inset the MBA 10 into a desired position in a bony segment.

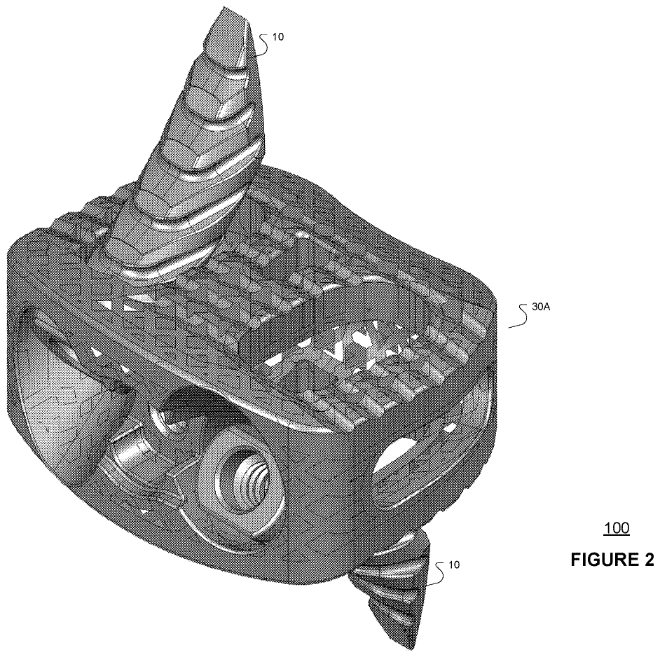

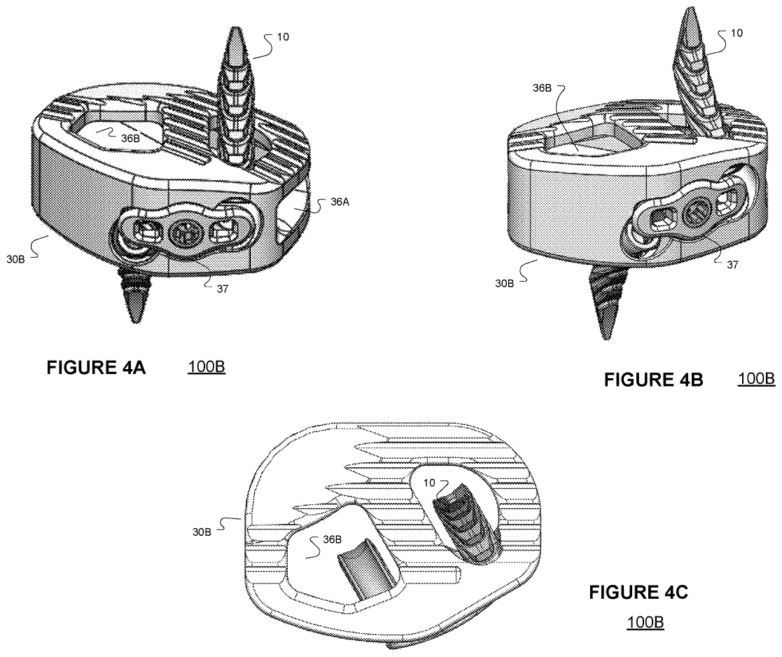

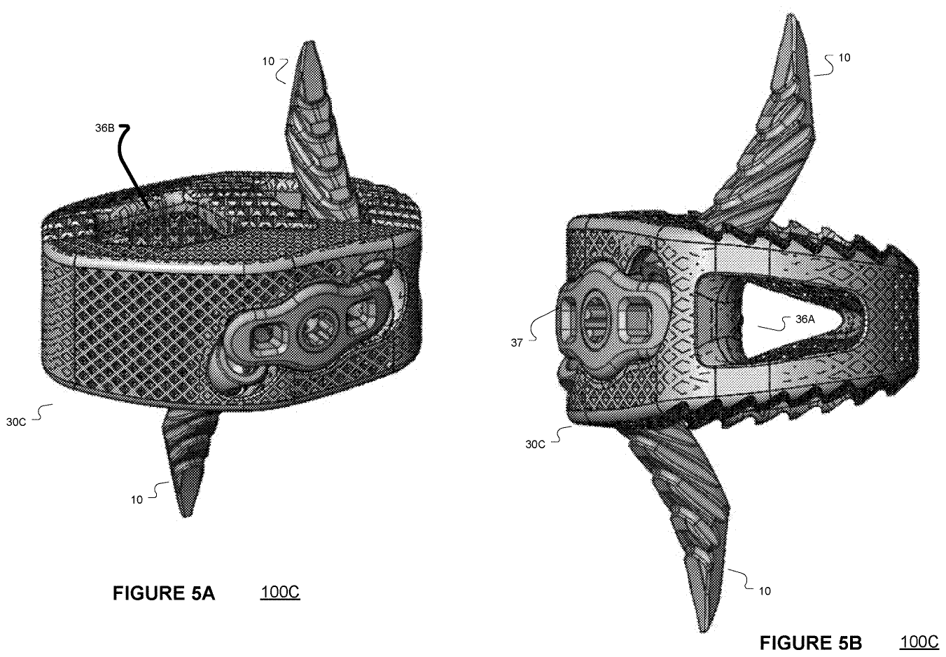

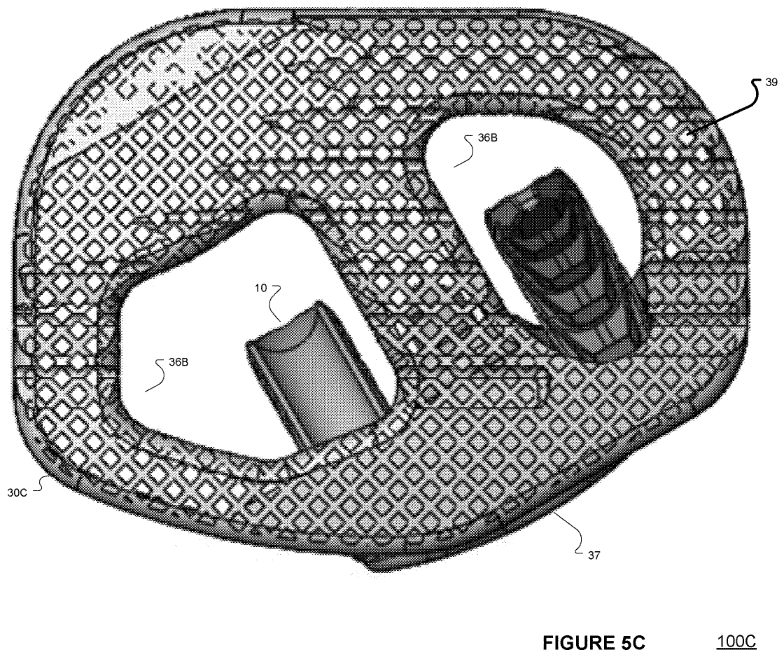

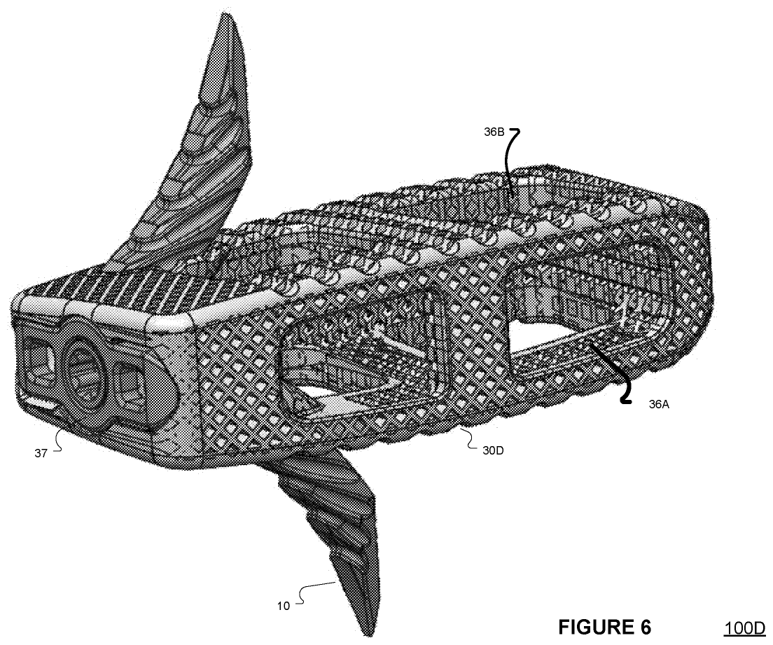

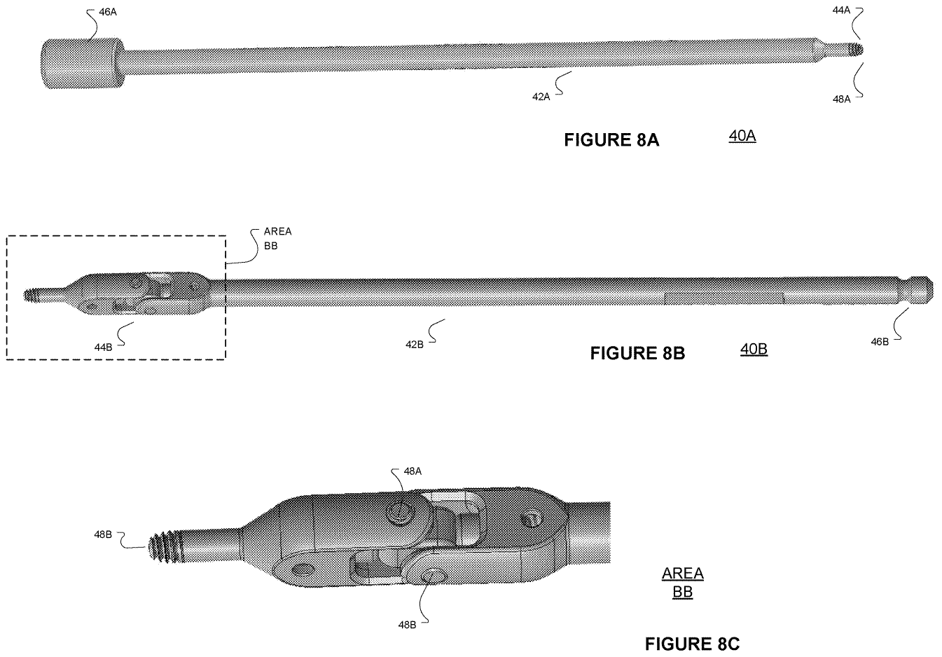

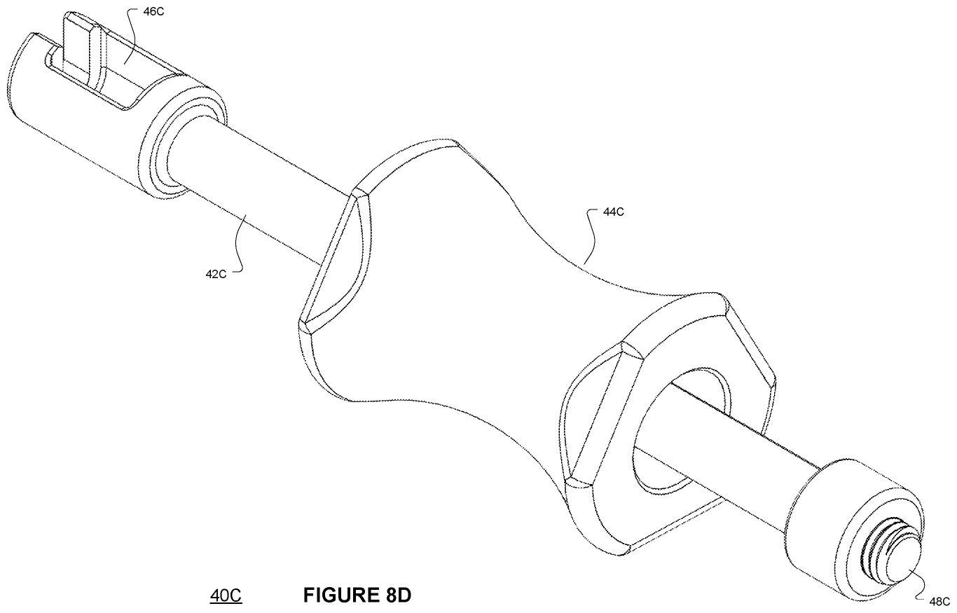

In an embodiment, an MBA 10 may be inserted into a desired position in a bony segment via an impaction tool that acts on the tool interface 16 flat impact surface 16A due to its configuration/geometry. As shown FIGS. 1A-1G, the tool interface 16 may include an internal threaded section 16B sized to receive a removal tool 40A, 40B, and 40C shown in FIGS. 8A, 8B, 8C, and 8D having interface's external thread 48A, 48B, and 48C as shown in FIGS. 8A-8D. The tool interface 16 may also include a chamfer 16C and counterbore 16D each having a diameter greater than the internal threads 16B. The base 14 may have a flat top and bottom section 14A and partially spherical sides 14B. This base 14 configuration enables the MBA 10 to have a large range of pivotability when used in conjunction with an implant 30A-D such as shown in FIGS. 2-6. As shown in FIGS. 2-6, an implant 30A-D may include a plurality of bone anchor interfaces 32 that are spherical in relief and enable the MBA 10 base 14 section to pivot over a large range. As shown in FIG. 2-6 an implant 30A-D may include a tool interface relief 34A, a tool interface internally thread bore 34B, a side fenestration 36A, and a top to bottom fenestration 36B where the fenestrations 36A, 36B may be packed or filled with an osteoconductive material prior to the implant 30A-D implantation between two, adjacent bony elements.

As shown in FIG. 2, when coupled to an implant, an MBA 10 may form about a 90-degree angle at its tip section 18 relative to the implant 30. When inserted between bony sections, each MBA 10 may provide substantial retention and anti-expulsion force with the bony segments. In an embodiment, the bony segments 52A-C may be vertebrae with disc nucleus 54A-B located between adjacent vertebrae 52A, 52B or 52B, 52C. In an embodiment, the vertebrae 52A-C may be lumbar, thoracic, or cervical vertebrae and an adjacent mammalian bony segments fixation system (AMBSFS) 100A-D (FIGS. 2-6) may be ideally inserted between two, adjacent vertebrae.

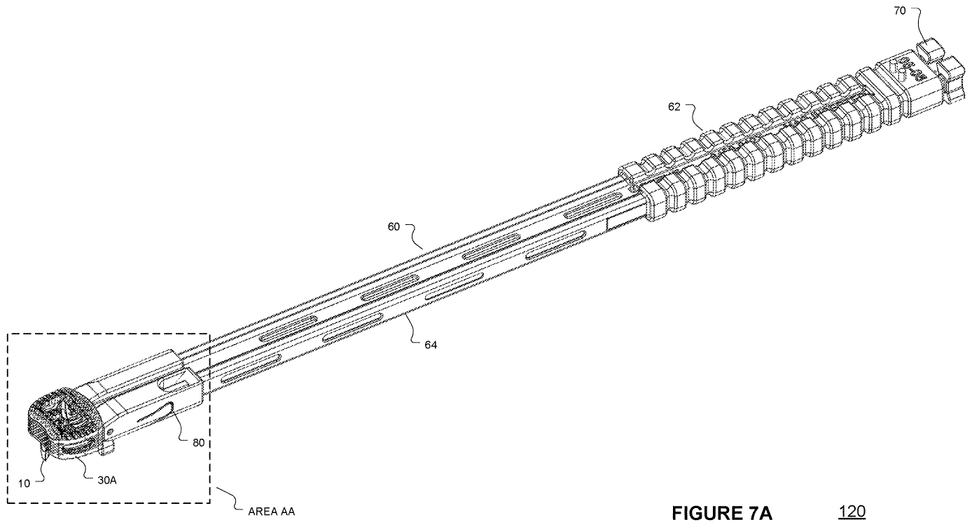

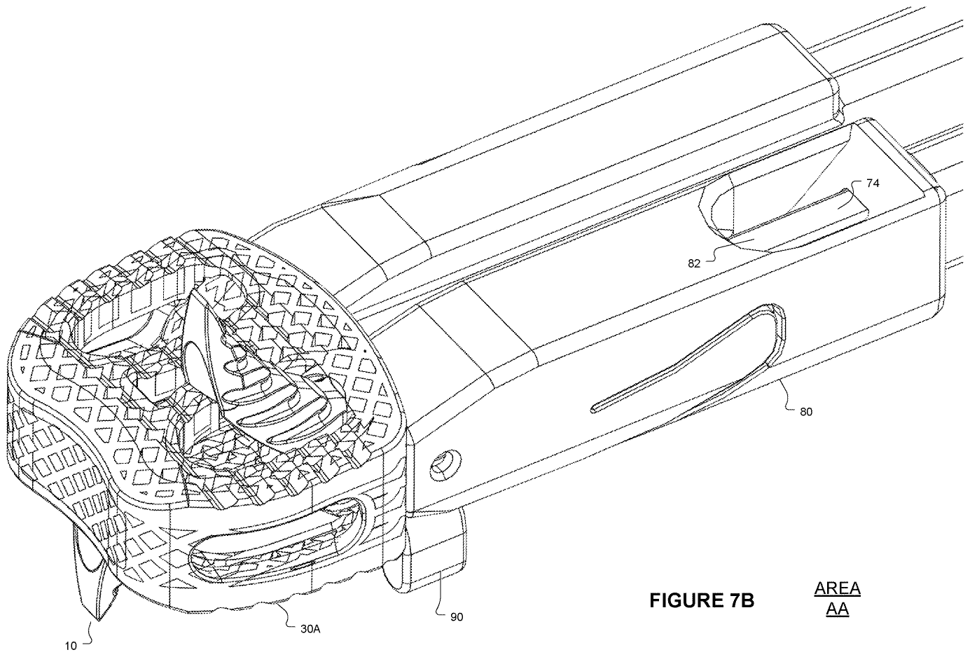

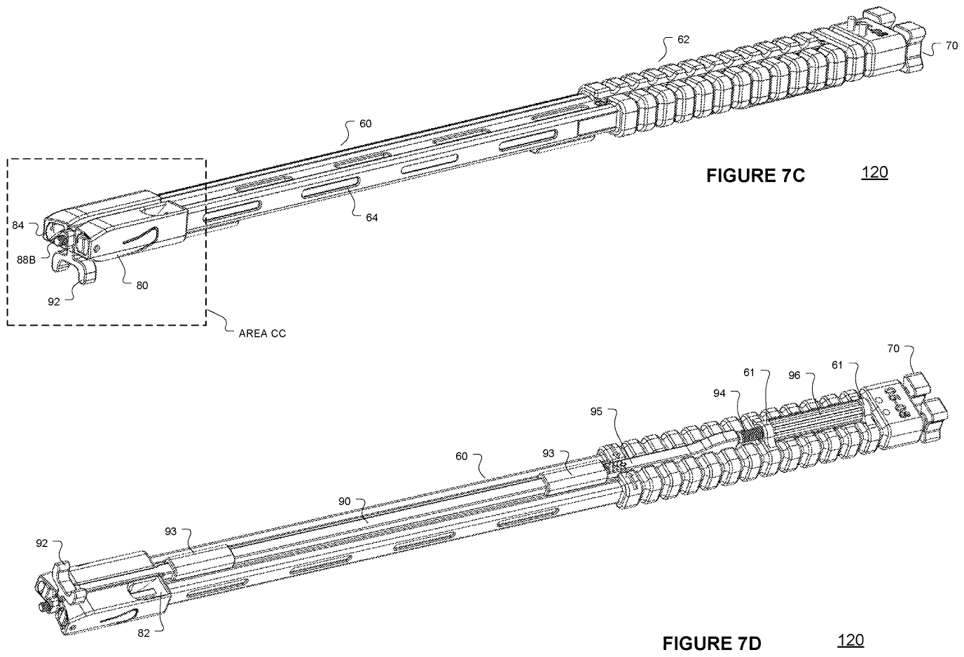

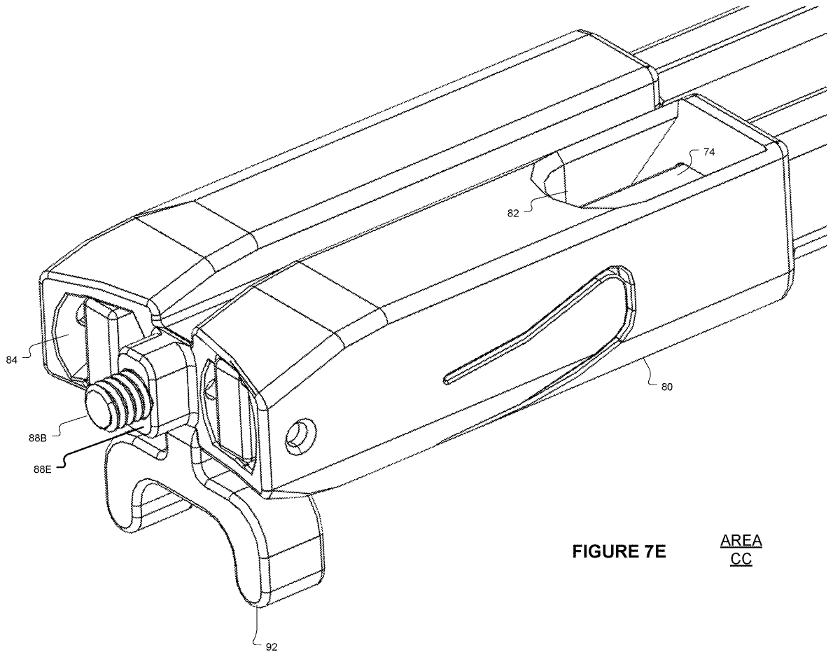

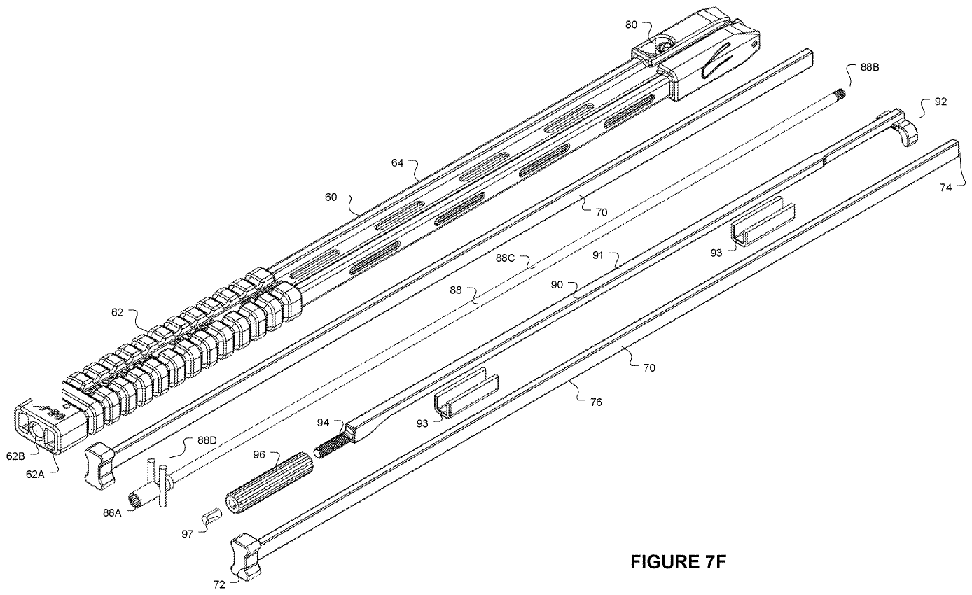

In an embodiment, an AMBSFS 100A-D may include an implant 30A-D and a plurality of MBA (anchors) 10. As described with reference to FIGS. 7A-7D, in an embodiment, a combination implant and anchor inserter (CIAIS) 120 may be employed by a user (such as a surgeon) to implant a AMBSFS between two, adjacent bony constructs. In an embodiment, the CIAIS 120 may store two MBA 10 of a AMBSFS 100A-100D in an implant and anchor insertion head (IAIH) 80 while simultaneously holding/being coupled to the AMBSFS implant 30A-D. The CIAIS 120 may further enable a User to first fixably place an implant 30A-D between adjacent bony segments (ABS) 52A, 52B including from an anterior position (part an anterior lumbar interior fusion (ALIF)) or cervical anterior system (30A), obliquely in an oblique lateral interbody fusion (OLIF) implant 30B and 30C, and laterally via a lateral lumbar interbody fusion (LLIF) implant 30D. The CIAIS 120 may second enable the User to fixably insert one or more MBA 10 through the implant 30A-D into one of the ABS 52A, 52B while the CIAIS 120 remains in the same location (adjacent the ABS 52A, 52B).

FIG. 7A is an isometric simplified drawing of a combination implant and anchor inserter system (CIAIS) 120 coupled to an implant 30A and anchors 10 according to various embodiments. FIG. 7B is an isometric simplified drawing of an Area AA shown in FIG. 7A according to various embodiments. FIG. 7C is an isometric simplified top drawing of the CIAIS 120 according to various embodiments. FIG. 7D is an isometric simplified bottom drawing of the combination implant and CIAIS 120 according to various embodiments. FIG. 7E is an isometric simplified enlarged drawing of Area CC shown in FIG. 7C according to various embodiments. FIG. 7F is an isometric simplified drawing of the removable elements 70, 88, 90 of the CIAIS 120 as shown in FIG. 7A according to various embodiments.

As shown in FIGS. 7A-7F, a CIAIS 120 may include a combination Implant and anchor inserter (CIAI) 60, several anchor impact elements (AIE) 70, an implant depth controller (IDC) 90, and an implant and anchor insertion head (IAIH) 80. As also shown in FIGS. 7A-7B, a CIAI 60 may include a user grip section 62 and a shaft 64 between the user grip section 62 and the IAIH 80. As shown in FIG. 7D, the IDC 90 may be located on the bottom of the CIAIS and control the position of its distal end 92 that is shaped to be placed adjacent to one of a pair of bony segments so that the implant 30A-D coupled to the IAIH 80 may inserted a known depth between the bony segments.

As shown in FIGS. 7D and 7F, the IDC 90 may include an elongated shaft 91 between a distal bony segment engaging end 92 and threaded proximal end 94. The threaded proximal end 94 may be threadably coupled to a rotatable cylinder 96 that is coupled the CIAIS 120 bottom via a coupler 97 that engages a section 61. Several sleeves 93 may also be coupled to the CIAI 60 where the sleeves 93 are shaped and sized to enable the elongated shaft 91 to freely slidably move therein. The shaft 91 may also include a depth scale 95 to help enable a User to select the depth past the end of the IAIH 80 an implant 30A-30D coupled thereto may extend into adjacent bony segments when the end 92 engages one of the adjacent bony segments.

In an embodiment, the anchor impact elements (AIE) 70 may include an impact head 72, an elongated shaft 76, and distal implant impact end 74. The head 72 has a flat proximal surface that may be impacted by an impact tool such as a hammer. The distal end 74 may be shaped to pass through segments 82 of the IAIH 80 to engage and drive an MBA 10 stored in the IAIH 80. The shaft length 76 may be selected to limit the overall passage of the distal end 74 within the IAIH 80 in an embodiment. The shaft 76 and distal end 74 may also be shaped to pass through CIAI user grip 62, grip openings 62A, and extension 64. Further, the CIAIS 120 may include a CIAI 60 and two AIE 70. The AIE 70 are shown fully inserted into the IAIH (head) 80 via the CIAI user grip 62 in FIG. 7C.

FIG. 7B is an isometric simplified drawing of area AA of the combination Implant and anchor inserter system (CIAIS) 120, in particular the IAIH 80 coupled to an implant 30A as shown in FIG. 7A according to various embodiments. In an embodiment, the IAIH 80 may be able to load, store, and enable a user to implant two MBA 10 and couple and enable a user to insert an implant 30A-D. As shown in FIGS. 7A-7F, a IAIH 80 may include two ports 82 sized to enable an MBA 10 to be inserted therein-one on top and on the bottom in an embodiment. The IAIH 80 ports 82 may also provide visualization and enable cleaning, the port 82 may allow a User to visualize the position of AIE 70 distal 74 and shaft 76 in an embodiment as shown in FIGS. 7A-7F. Further, IAIH 80 ports 82 may enable a user to eject or insert an MBA 10 for implantation therein. In an embodiment the IAIH 80 includes implantation ports 84 are sized to enable an anchor 10 to pass therethrough and configured to align with an implant's 30A-D MBA anchor interfaces 32 when the implant 30A-D is coupled to the IAIH 80.

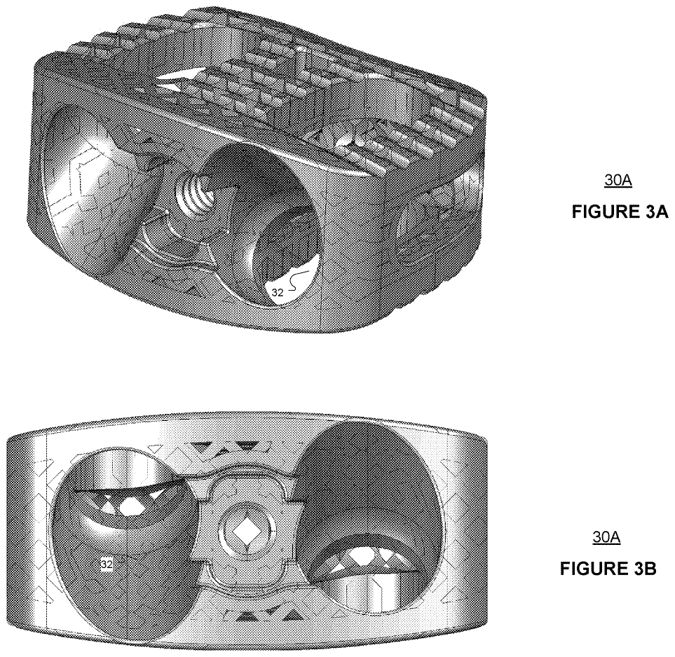

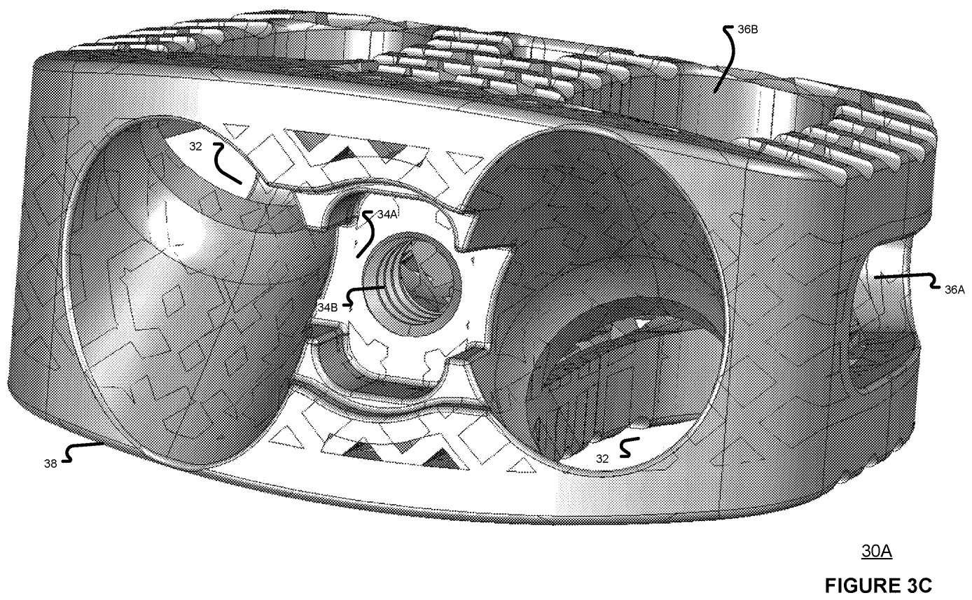

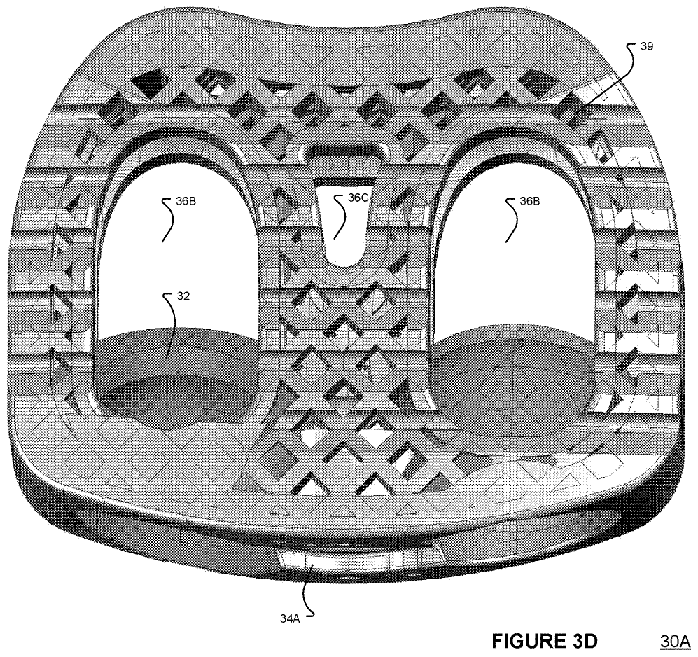

FIG. 7E is an isometric simplified enlarged drawing of area CC of the combination Implant and anchor inserter system (CIAIS) 120 as shown in FIG. 7C according to various embodiments. As shown in FIG. 7E, the IAIH 80 includes external threads 88B shaped and sized to engage the internal threads 34B on an implant 30A such as shown in FIG. 3C. The IAIH 80 also includes a shaped protrusion 88E that is shaped to securely fit within the relief 34A of an implant such as implant 30A shown in FIG. 3C. The combination of the threads 88B and protrusion 88E enable the IAIH 80 to be securely coupled to an implant 30A-D for implantation between adjacent bony segments.

In an embodiment as shown in FIGS. 7A-7F and in particular 7F, an IAIH 80 may further include an implant connection system ICS 88 with a threaded implant 30A-D interface 88B that is configured to be threadably secured and removed to an implant's 30A-D tool interface internally thread bore 34B (FIG. 3C). The threaded implant 30A-D interface 88B may enable a User to securely couple an implant the CIAI 60 IAIH 80 and securely insert the implant 30A-D between adjacent bony elements.

FIG. 7F is a simplified exploded side view of a CIAIS 120 shown in FIGS. 7A-7D according to various embodiments. As shown in FIG. 7F, the CIAI 60 may include a user grip 6F2, shaft 64, two AIE 70, IAIH 80, ICS 88, and IDC 90. In an embodiment, an ICS 88 may include a tool interface 88A, implant 30A-D bore 34B bore-interface 88B, elongated circular shaft 88C, and connection/retention pins 88D. The grip section 62 also includes port 62B that is sized to enable the ICS 88 proximal shaft section 88C to pass therethrough. Further, the grip section 62 may include ports 62A that are sized to enable an AIE 70 proximal end 74 and shaft 76 to pass therethrough.

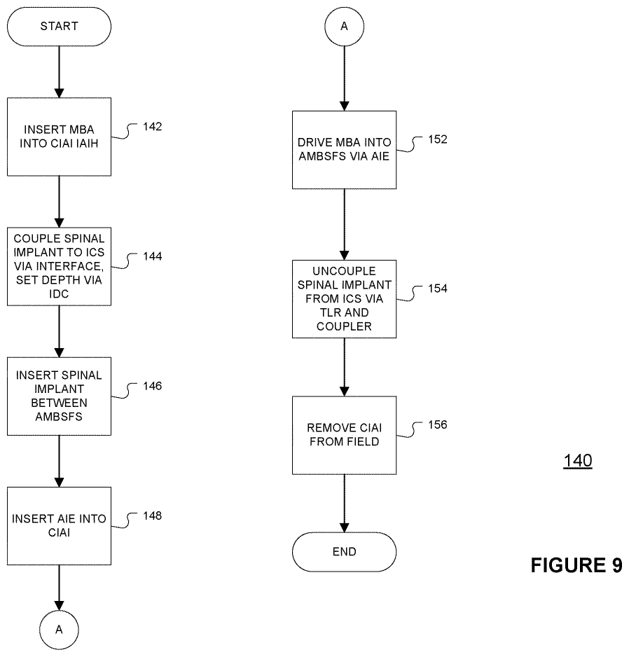

Via the CIASIS 120, an implant 30 and two MBA 10 may be inserted between two, adjacent bony segments. FIG. 9 is an algorithm 140 for employing an CIASIS 120 to insert an AMBSFS including an implant 30A-D and two MBA 10 between ABS. In an embodiment, one or more of MBA 10 of a AMBSFS may be inserted into the CIAIS 120 IAIH 80 (activity 142). Then the combination implant and anchor inserter (CIAI) 60 may be employed to couple the IAIH 80 to an implant 30A-D and insertion depth configured via IDC 90 by rotating cylinder 96 per activity 144 of algorithm 140 according to various embodiments. The TLR 90 may be operatively coupled to the ICS 88 so the ICS 88 implant interface 88B may securely couple with the implant 30A-D threaded bore 34B.

Then the combination implant and anchor inserter (CIAI) 60 coupled to an implant 30A-D with mammalian bony anchors 10 stored therein prior to implant insertion may be inserted between adjacent bony segments AMBSFS per activity 146 according to various embodiments. In an embodiment, a hammer or mallet may be applied to the CIAI 60 user grip 62 proximal end to advance an implant between adjacent bony segments AMBSFS. It is noted that IDC 90 distal end 92 may be shaped to engage registration elements already engaging a bony segment of the adjacent bony segments in an embodiment.

One or both AIE 70 may be inserted into a ports 62A of CIAI 60 until the AIE 70 distal section 74 ends engages a MBAs 10 stored in the IAIH 80 per activity 148 of algorithm 140. Then by advancing the AIE 70 flat section 72 to the CIAI 60 user grip 62 proximal end, an MBA 10 may be inserted through an implant 30A-D port 32 and into one of the adjacent bony segments per activity 152 of algorithm 140. In an embodiment, a hammer or mallet may be applied to the AIEs 70 flat heads 72 to advance the MBAs 10 through an implant 30A-D and into the one of the two adjacent bony segments. In an embodiment, one or both MBA 10 may be inserted at a time by only advancing one or both AIE 70.

Then a tool may be operatively coupled to the ICS 88 tool interface 88A so the ICS 88 implant interface distal end 88B may be decoupled from an implant's 30A-D threaded bore 34B per activity 154 of algorithm 140. Then the CIAIS 120 with AIE 70 may be removed from the field per activity 156 of algorithm 140.

When necessary to remove an inserted MBA 10, it may be ideally moved along the arc of the shaft 12 radius 17A shown in FIG. 1E. The removal tools 40A, 40B, and 40C shown in FIGS. 8A-8D may be coupled via the tool's external threads 48A, 48B, and 48C to an inserted MBA 10 tool interface 16 internal threads 16B and be guided to the threads 16B via the MBA's 10 chamfer 16C and counterbore 16D. The tools 40A, 40B, and 40C may then be used to remove an inserted MBA 10 at or near the arc 17A via the shafts 42A, 42B, and 42C and handles 46A, 46B, and 46C. As shown in FIG. 8C, a tool 40B may be pivotable about two different axes 48A, 48B to ease the attachment to an inserted MBA 10 to be removed along the arc or radius 17A. The slap tool 40C shown in FIG. 8D may be coupled to an MBA 10 to aid its removal or initial movement from a bony segment.

As noted, the geometry of an MBA 10 may provide greater expulsion strength and reliable cortical vertebral endplate penetration when inserted into vertebra in an embodiment. In an embodiment, the MBA 10 outer surface may have scaling to provided increased osteointegration. The MBA 10 scallops 12A and undercuts 12B may also grip bone when inserted into thereto. The MBA 10 tip 18 structure may enable it to reliably penetrate cortical vertebral endplates without causing nor incurring fracture damage when be inserted into a vertebra. In an embodiment, the MBA 10 may provide various fixation angles, convergent and/or divergent. In an embodiment, the MBA 10 and implant 30A-D may be formed of a biocompatible, substantially radio-lucent material or complex of materials.

In an embodiment, an implant 30A-D and MBA 10 may be formed of a metal, alloy, combination thereof, a polymer, a ceramic, or combination of both, including Polyether ether ketone (PEEK) or other member of the polyaryletherketone family or other osteoconductive material. In an embodiment, an MBA 10 and implant 30A-D may be formed from Titanium.

The accompanying drawings that form a part hereof show, by way of illustration and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In the foregoing Detailed Description, various features are grouped together in a single embodiment for the purpose of streamlining the disclosure. This method of disclosure is not to be interpreted to require more features than are expressly recited in each claim. Rather, inventive subject matter may be found in less than all features of a single disclosed embodiment. Thus, the following claims are hereby incorporated into the Detailed Description, with each claim standing on its own as a separate embodiment.

What is claimed is:

1. A combination implant and anchor inserter (CIAIS) that enables a user to insert an adjacent mammalian bony segments fixation system (AMBSFS) between first and second adjacent bony segments, the AMBSFS including an implant and at least two mammalian bony anchors (MBA) each MBA having an overall length and effective radius (MBA radius) along its overall length, the implant including MBA interfaces for the two mammalian bony anchors, the CIAIS including:
an implant and anchor insertion head (IAIH), the IAIH including:
an implant connection system (ICS), the ICS couplable to implant to be fixably inserted between the first and the second adjacent bony segments;
a first insertion port sized and having a curvature similar the MBA radius to enable a first MBA to be inserted and stored therein;
a second insertion port sized and having a curvature similar the MBA radius to enable a second MBA to be inserted and stored therein;
a first ejection port coupled to the first insertion port, the first ejection port aligned with a first MBA interface of the implant when the implant is coupled to the ICS and having a curvature similar the MBA radius to enable an MBA to transit therethrough at a radial path; and
a second ejection port coupled to the second insertion port, the second ejection port aligned with a second MBA interface of the implant when the implant is coupled to the ICS and having a curvature similar the MBA radius to enable an MBA to transit therethrough at a radial path,
wherein the first MBA and the second MBA may be inserted at the same time in the first and the second adjacent bony segments when the implant is fixably inserted between the first and the second adjacent bony segments.

2. The CIAIS of claim 1, wherein the CIAIS enables a user to first fixably insert the implant between the first and the second adjacent bony segments and then simultaneously deploy a first MBA to be couple the implant to the first bony segment in an upward curved direction and a second MBA to be couple the implant to the second bony segment in an downward curved direction.

3. The CIAIS of claim 1, wherein the implant includes an internally threaded bore on its rear section and the ICS includes threaded interface configured to be threadably securable and removable with the implant internally threaded bore.

4. The CIAIS of claim 3, wherein the implant includes a recess on its rear section leading to the internally threaded bore and the ICS includes an implant engagement protrusion shaped in relief of the implant recess and configured to nest therein when the implant is threadably coupled to the ICS threaded interface.

5. The CIAIS of claim 1, wherein the first ejection port has an upward curvature similar the MBA radius to enable an MBA to transit therethrough at an upward radial path.

6. The CIAIS of claim 5, wherein the second ejection port has a downward curvature similar the MBA radius to enable an MBA to transit therethrough at an downward radial path.

7. The CIAIS of claim 6, wherein the implant includes an upward curving MBA interface for a mammalian bony anchor and the first ejection port directs the MBA into the implant upward curving MBA interface.

8. The CIAIS of claim 7, wherein the implant includes an downward curving MBA interface for a mammalian bony anchor and the second ejection port directs the MBA into the implant downward curving MBA interface.

9. The CIAIS of claim 1, the CIAIS further including a first and a second MBA impact elements (AIE), each AIE including:
a head;
a distal section; and
an elongated shafted between the head and the distal section,
wherein the distal end is shaped to extend into one of the first and the second insertion port and engage an MBA base when placed adjacent thereto and wherein the first and the second AIE may be engaged at the same time to each advance a respective MBA.

10. The CIAIS of claim 9, wherein AIE distal section includes:
a proximal portion; and
a distal portion,
wherein the proximal portion has a larger cross-sectional area than the distal portion.

11. The CIAIS of claim 10, wherein the distal end is shaped to extend into one of the first and the second insertion port and engage an MBA base when placed adjacent thereto and wherein the first and the second AIE may be engaged at the same time to each advance a respective MBA at a radial path.

12. The CIAIS of claim 11, wherein the AIE distal portion is substantially rectangular in cross-section.

13. The CIAIS of claim 12, wherein the AIE flat, upper distal end portion may engage an MBA base when placed adjacent thereto.

14. The CIAIS of claim 11, wherein the insertion ports are sized to enable a first and a second AIE distal section distal portion to extend thereinto and engage a first and second MBA stored therein at the same time, while limiting the travel of the AIE with the IAIH.

15. The CIAIS of claim 12, wherein the insertion ports are sized to enable a first and a second AIE distal section distal portion to extend thereinto and engage a first and second MBA stored therein, while limiting the travel of the AIE distal section proximal portion therein.

16. The CIAIS of claim 14, wherein each AIE head has flat portion perpendicular to the AIE elongated shaft.

17. The CIAIS of claim 15, wherein each AIE head has flat portion perpendicular to the AIE elongated shaft.

18. The CIAIS of claim 17, wherein the CIAIS enables a user to first fixably insert the implant between the first and the second adjacent bony segments and then simultaneously deploy a first MBA to be couple the implant to the first bony segment and a second MBA to be couple the implant to the second bony segment via a first AIE and second AIE.
